(19) United States
(12) Patent Application Publication
(10) Pub. No.: US 2004/0151769 A1
(43) Pub. Date: Aug. 5, 2004

(54) Film coated tablet containing an extract of red vine leaves

(75) Inventors: Anke Esperester, Mainz (DE); Fritz Sacher, Gau-Algesheim (DE); Eckhard Schaefer, Heusenstamm (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/743,170

(22) Filed: Dec. 22, 2003

Related U.S. Application Data
(60) Provisional application No. 60/499,530, filed on Sep. 2, 2003.

(30) Foreign Application Priority Data
Dec. 31, 2002 (EP) 02 029 108
Sep. 5, 2003 (EP) 02 019 636

(51) Int. Cl. A61K 9/20; A61K 35/78

(52) U.S. Cl. 424/465; 424/774

(57) ABSTRACT

A film coated tablet comprising:

(a) at least 50% by weight of a dried aqueous extract of red vine leaves;

(b) up to 50% by weight of an excipient consisting essentially of: at least one binder, at least one disintegrant, at least one filler, and a lubricant; and

(c) a tablet film consisting essentially of: a film former, a plasticizer, a coating agent, and optionally a coloring agent,

and methods of making and using the same.

### RELATED APPLICATIONS

This application claims priority to U.S. Serial No. 60/499,530, filed Sep. 2, 2003, European Patent Application No. 02 029 108.4 filed Dec. 31, 2002, and European Patent Application No. 02 019 636.4 filed Sep. 5, 2003, each of which is hereby incorporated by reference in its entirety.

### FIELD OF THE INVENTION

The invention relates to a film coated tablet comprising a dried extract of red vine leaves, an excipient and a tablet film and the use thereof for the improvement of the blood circulation and/or the oxygen supply of the lower extremities.

### BACKGROUND OF THE INVENTION

Chronic venous insufficiency (CVI) is a progredient disease and will lead in many patients, especially if untreated, to edema, coronal phlebectasia (Widmer stage I), hyperpigmentation, induration, lipodermatosclerosis, white atrophy (Widmer stage II), or varicose leg ulcers (Widmer stage III). Chronically disturbed hemodynamics of deep or superficial veins due to obstructed venous segments or valvular incompetence lead usually to skin diseases in the inner ankle area of the lower limbs. Disturbances in the microcirculation of the skin have been considered to be major contributors for skin changes associated with chronic venous hypervolaemia and venous hypertension (e.g., B. Fagrell, Vital Microscopy and the Pathophysiology of Deep Venous Insufficiency, Int. Angiol. 1995, 14:18-22.; M. Jünger, T. Klyscz, M. Hahn, and G. Rassner, Disturbed Blood Flow Regulation in Venous Leg Ulcers, Int. J. Microcirc. 1996, 16:259-265).

Obviously, cutaneous microangiopathy of clinical relevance such as enlarged, tortuous capillaries surrounded by micro-edema contributes to the skin alterations in the lower limbs and determines the course of CVI (B. Fagrell, loc. cit. and M. Jünger et al., loc. cit.). The application of the laser Doppler technique in venous disorders is well-illustrated (e.g., I. I. Tulevski, D. T. Ubbink, and M. J. H. M. Jacobs, Red and Green Laser Doppler Compared with Capillary Microscopy to Assess Skin Microcirculation in the Feet of Healthy Subjects, Microvasc. Res. 1999, 58(2):83-88; A. Bollinger, K. Jäger, M. Jünger, and H. Seifert, The Vascular Laboratory: Advances in Non-Invasive Techniques, World J. Surg. 1988, 12:724-731).

Different techniques have been developed to investigate microcirculation in both functionally different layers of the skin: the deeper, mainly thermoregulatory layer and the superficial, nutritive layer. Microcirculatory disturbances in the superficial nutritive layer are of utmost relevance for trophical skin changes (M. Jünger et al., loc. cit. and M. E. Gschwandtner, E. Ambrozy, S. Fasching, A. Willfort, B. Schneider, and K. Böhler et al., Microcirculation in Venous Ulcers and Surrounding Skin: Findings with Capillary Microscopy and Laser Doppler Imager, Eur. J. Clin. Invest. 1999, 29:708-716).

The British patent GB 934,554 discloses that the capillary resistance of guinea pigs deficient in a vitamin can be enhanced by intraperitonal administration of an alcoholic extract of vine leaves.

The International Patent Application WO 01/28363 discloses a method for preventing or alleviating the discomfort associated with mild-to-moderate chronic venous insufficiency of the lower extremities with the aid of an aqueous extract of red vine leaves. In addition, a daily dosage regimen of 80 mg to 1000 mg divided up in 1 to 3 capsules is suggested.

The problem underlying the present invention was to provide a dosage form which allows to administer such high amounts of aqueous extract of red vine leaves in the suggested regimen. In view of patient compliance, such dosage forms should not be too big, in order to facilitate swallowing. On the other hand, the dosage form must have a high stability to ensure long shelf storage times. Moreover, high bioavailability is but a prerequisite for the therapeutic and/or preventive success of such a dosage form.

### SUMMARY OF THE INVENTION

It has been surprisingly found that a film coated tablet comprising:

(a) at least 50% by weight of a dried extract of red vine leaves, which is obtainable by extraction of red vine leaves with water and drying;

(b) up to 50% by weight of an excipient consisting essentially of: at least one binder, at least one disintegrant, at least one filler, and a lubricant; and

(c) a tablet film consisting essentially of: a film former, a plasticizer, a coating agent, and optionally a coloring agent,

fulfils these requirements and can be used to significantly enhance the microcirculation and the oxygen supply at the predominantly affected perimalleolar area of the leg in CVI patients.

Accordingly, the invention relates to a film coated tablet comprising the following constituents:

(a) at least 50% by weight of a dried extract of red vine leaves, which is obtainable by extraction of red vine leaves with water and drying;

(b) up to 50% by weight of an excipient consisting essentially of: at least one binder, at least one disintegrant, at least one filler, and a lubricant; and

(c) a tablet film consisting essentially of a film former, a plasticizer, a coating agent, and optionally a coloring agent.

Another aspect of the present invention is a process for preparing such a film coated tablet comprising the steps of:

(A) mixing the dried aqueous extract of red vine leaves (a) with the excipients (b), optionally in the presence of a volatile diluent;

(B) optionally screening the mixture obtained;

(C) compressing the mixture with a suitable tablet press; and

(D) coating the resulting tablet with the tablet film (c).

Furthermore, the invention relates the use of such a film coated tablet for preparing a pharmaceutical or dietary composition for the treatment or prevention of the discomfort, disorder, and/or disease associated with chronic venous hypervolaemia and venous hypertension.

Furthermore, the invention relates to an aqueous extract of red vine leaves, which is obtainable by a method comprising the steps of:

(a) collecting red vine leaves at a point of time when the content in flavonoids has reached an optimum;

(b) drying and crushing the leaves;

(c) cutting the leaves to pieces;

(d) extracting the leaves with water at elevated temperatures for 6 to 10 hours;

(e) concentrating and drying the obtained extract; and

(f) addition of up to 10% by weight of silica relating to the final total amount of the resulting extract.

### BRIEF DESCRIPTION OF THE DRAWINGS

FIG. 1 shows the schematic design of the clinical study carried out.

FIG. 2 shows the influence of the vine leaf extract

[symbol] AS 195 (360 mg) compared with

[symbol] placebo

on the microcirculation measured with Laser Doppler flowmetry (LDF 10-37 kHz).

FIG. 3 shows the influence of the vine leaf extract

[symbol] AS 195 (360 mg) compared with

[symbol] placebo

on transcutaneous oxygen partial pressure (tcO₂).

### DETAILED DESCRIPTION OF THE INVENTION

The composition of the present invention preferably consists of herbal ingredients derived by an aqueous extraction from red vine leaves (folia vitis viniferae; Extractum Vitis viniferae e folium spissum et siccum) and an acceptable carrier. This extract contains flavon(ol)-glycosides, -glucuronides and flavonoids, with quercetin-3-O-β-D-glucuronide and isoquercitrin (quercetin-3-O-β-glucoside) as its main active ingredients. The range of their pharmacological actions has not yet been fully elucidated, but in vitro studies indicate that they have antioxidant and anti-inflammatory properties and that they inhibit platelet aggregation and hyaluronidase and reduce edema, possibly by reducing capillary permeability. Preclinical in vivo experiments demonstrated anti-inflammatory and capillary wall thickening effects.

The film coated tablet according to the present invention comprises 50% to 70% of a dried aqueous red vine leaf extract with a high flavonoid content of 2% to 15%.

As a rule, the relation by weight between the dried extract and the excipients used to produce the core of the tablet is between 1:1 to 2:1, preferably between 1.1:1 and 1.8:1, in particular, between 1.25:1 and 1.75:1.

A film coated tablet comprising:

(a) 50 to 70% by weight of the dried extract of red vine leaves;

(b) 25 to 49% by weight of the excipient; and

(c) 1 to 5% by weight of the tablet film,

based on the total mass of the film coated tablet, is preferred.

More preferred is a film coated tablet comprising:

(a) 51% to 59%, in particular about 55% by weight of the dried extract of red vine leaves;

(b) 38% to 48%, in particular about 43% by weight of the excipient; and

(c) 1% to 3%, in particular about 2.7% by weight of the tablet film,

based on the total mass of the film coated tablet.

Another preferred embodiment is a film coated tablet according, wherein the excipient (b) consists essentially of: 70% to 85% by weight of at least one binder, 0.5% to 12.5% by weight of at least one disintegrant, 5% to 15% by weight of at least one filler, and 1% to 5% by weight of at least one lubricant, based on the total mass of the combined excipients.

The term “binder” a used hereinbefore and hereinafter denotes an excipient which is suitable for binding other components to one another. Preferred binders according to the invention are selected from among: powdered cellulose, microcrystalline cellulose, sorbitol, starch, polyvinylpyrrolidone (povidone), copolymers of vinylpyrrolidone with other vinyl derivatives (copovidone), cellulose derivatives, particularly methylhydroxypropylcellulose, e.g., METHOCEL® A15LV, and mixtures of these compounds. The preferred binders are powdered cellulose, particularly microcrystalline cellulose and/or copovidone. If the abovementioned binders are used, the amount by weight, based on the total mass of the tablet according to the invention, is preferably in a range of 15 wt. % to 45 wt. %, more preferably 25 wt. % to 40 wt. %, most preferably about 33 wt. %. Thanks to the particularly preferred binder microcrystalline cellulose, tablets are obtained having a high stability and good compliance for the patients to whom the aqueous extract of red vine leaves has to be administered.

The tablet according to the invention also contains disintegrants in addition to the abovementioned ingredients. Within the scope of the present invention, these disintegrants may optionally also be known as breakdown agents. These are preferably selected, according to the invention, from among sodium starch glycolate, crosslinked polyvinylpyrrolidone (crospovidone), croscarmellose sodium salt (sodium salt of cellulose carboxymethyl ether, crosslinked), sodium-carboxymethylcellulose, dried maize starch, colloidal anhydrous silica, and mixtures thereof. Within the scope of the present invention it is particularly preferred to use sodium starch glycolate, crospovidone and, preferably, the sodium salt of crospovidone or croscarmellose and colloidal anhydrous silica. Most preferred is a mixture of croscarmellose sodium, colloidal anhydrous silica, and optionally crospovidone. If the abovementioned disintegrants are used, the amount by weight, based on the total mass of the tablet according to the invention, is preferably in a range of about 0.5 wt. % to 10 wt. %, more preferably, about 1.5 wt. % to 7.5 wt. %. Thanks to the particularly preferred combination of disintegrants, tablets are obtained having a high stability and provide high bioavailability to the aqueous extract of red vine leaves.

The tablet according to the invention also contains a filler. As a rule, fillers are inert compounds such as inorganic metal oxides or inorganic phosphate or hydrogen phosphate. Preferably the filler is anhydrous calcium hydrogen phosphate. If the abovementioned fillers are used, the amount by weight, based on the total mass of the tablet according to the invention, is preferably in a range of about 1 wt. % to 10 wt. %, more preferably about 2 wt. % to 8 wt. %.

The tablet according to the invention also contains flow agents or flow regulators and also lubricants, as additional ingredients. These include, within the scope of the present invention, for example, silicon dioxide, talc, stearic acid, sodium stearyl fumarate, magnesium stearate, and glycerol tribehenate. According to the invention, magnesium stearate is preferably used. If the abovementioned preferred lubricants are used, the amount by weight thereof, based on the total mass of the tablet according to the invention, is preferably in a range of about 0.1 wt. % to 10 wt. %, preferably about 0.5 wt. % to 5 wt. %, more preferably between 0.6 wt. % and 1.5 wt. %.

Furthermore, preferred is a film coated tablet, wherein the tablet film (c) consists essentially of 50% to 85% by weight of at least one film former, 5% to 10% by weight of at least one plasticizer, 10% to 20% by weight of at least one coating agent such as talc, and 0% to 15% by weight of at least one colorant, based on the total mass of the tablet film (c).

The tablet film according to the invention may also contain one or more synthetic or natural, pharmaceutically acceptable colorant, preferably one or more inorganic metal oxides such as titanium dioxide (E171) and/or ferric oxide (E172). If the abovementioned preferred colorants are used, the amount by weight thereof based on the total mass of the tablet according to the invention is 0.01 wt. % to 0.5 wt. %.

It is a further object of the present invention to provide a film coated tablet for preventing and/or alleviating the discomfort associated with mild-to-moderate chronic venous insufficiency of the lower extremities comprising herbal ingredients, wherein the tablet is manufactured pursuant to a controlled process that preserves the herbal curing qualities of the ingredients.

It is still a further object of the present invention to provide a film coated tablet which is effective in preventing and/or alleviating the discomfort associated with mild-to-moderate chronic venous insufficiency of the lower extremities.

It is still a further object of the present invention to provide a film coated tablet for preventing and/or alleviating the discomfort associated with mild-to-moderate chronic venous insufficiency of the lower extremities comprising herbal ingredients and having minimal or no side effects, thus being safe for internal consumption and a high stability and good patient compliance.

The aqueous extract prepared from dried red vine leaves is characterized by a high content of 2% to 20%, preferably 2% to 10%, of biologically active flavonoids.

The term “a person in need thereof” or “patient” as used hereinabove and hereinbelow relates to a female or male person who suffers from clinically not relevant early stages of chronic venous insufficiency (CVI) from proven CVI stage I and II according to Widmer. As a rule, such patients are elderly people with an age of between 30 and 80, preferably between 32 and 76 years having an mean age (±standard deviation) of 55.2±7.7 years. As a rule CVI is more expressed in female than in male patients.

In order that this invention be more fully understood, the following examples are set forth. These examples are for the purpose of illustrating embodiments of this invention, and are not to be construed as limiting the scope of the invention in any way. The examples which follow are illustrative and, as recognized by one skilled in the art, particular conditions could be modified as needed for individual compositions. Materials used in tests below are either commercially available or easily prepared from commercially available materials by those skilled in the art.

The basis of the tablet is the aqueous extract of red vine leaves (foliae vitis viniferae L.). The starting material for the preparation of the extract are red vine leaves collected at a point of time where the content in flavonoids has reached an optimum. This is usually the case around the harvesting time of the grapes. The leaves are carefully dried and crushed. For extraction, the leaves are cut to pieces of preferably 5 mm to 10 mm. To achieve a high content in flavonoids, the extraction is done at elevated temperature, preferably at a temperature in the range of 60° C. to 80° C., over a time of at least 6 up to 10 hours. The preferred method is that of an exhaustive percolation.

The so-called fluid extract obtained in the course of the extraction is concentrated by use of a suitable evaporator. The thick extract obtained in this step is dried, for instance, by use of a vacuum drying oven or a vacuum drying conveyer.

All or some of the excipients may be added during drying to facilitate further processing of the extract. As a rule, up to 10% of one or more constituents of the excipients can be added during the drying process.

Preferably a part of the flow regulator such as colloidal, anhydrous silica is added to the extract during drying or before admixing with the other constituents. Preferably the resulting extract composition contains 0.5% to 10% by weight, in particular 2.5% to 7.5% by weight, most preferably about 4% by weight of colloidal, anhydrous silica.

Surprisingly, tablets obtained from an extract to which a part of the excipients have been added during the drying process show an enhanced stability.

Most preferably the film coated tablet according to this invention consists of

300 to 500 mg, preferably 320 to 400 mg, in particular about 355 to 380 mg, of dry aqueous extract of red vine leaf (4-6:1) (extractum vitis viniferae foliae aquosum siccum), which may contain up to 10% weight of a flow regulator, in particular colloidal, anhydrous silica;

the following excipients of the tablet core: microcrystalline cellulose, croscarmellose sodium, calcium hydrogen phosphate (anhydrous), colloidal silica (anhydrous), or magnesium stearate, and optionally crospovidone, and

a tablet film consisting of: hypromellose, glyceryl tristearate, titanium dioxide (E 171), talc, or ferric oxide, red (E 172).

Film coated tablets were prepared with the ingredients listed in the following Tables A and B.

TABLE A

| | Quantity per Film | |
|---|---|---|
| | Coated Tablet | |
| Name of Ingredient | [mg/658.000 mg] | Function |
| Tablet Core: | | |
| Vitis viniferae folium dry | 360.000 | Active ingredient |
| extract aqueous (4:1 to 6:1) | | |
| microcrystalline cellulose | 219.000 | Binder, disintegrant |
| croscarmellose sodium | 18.000 | Disintegrant |
| calcium hydrogen | 30.000 | Filler |
| phosphate, anhydrous | | |
| silica, colloidal, anhydrous | 4.000 | Flow regulator, |
| | | disintegration accelerator |
| magnesium stearate | 9.000 | Lubricant |
| Tablet Film: | | |
| hypromellose | 11.383 | Film former |
| glyceryl tristearate | 1.138 | Plasticizer |
| titanium dioxide (E 171) | 0.783 | Coloring agent |
| talc | 3.131 | Coating agent |
| ferric oxide, red (E 172) | 1.565 | Coloring agent |

The extract is mixed with the excipients of the tablet core and compressed on a suitable tablet press.

TABLE B

| | Quantity per Film | |
|---|---|---|
| | Coated Tablet | |
| Name of Ingredient | [mg/658.000 mg] | Function |
| Tablet Core: | | |
| Vitis viniferae folium dry | 360.000 | Active ingredient |
| extract aqueous (4:1 to 6:1) | | |
| silica, colloidal, anhydrous | 15.000 | binder |
| microcrystalline cellulose | 214.000 | Binder, disintegrant |
| croscarmellose sodium | 18.000 | Disintegrant |
| calcium hydrogen | 30.000 | Filler |
| phosphate, anhydrous | | |
| silica, colloidal, anhydrous | 6.000 | Flow regulator, |
| | | disintegration accelerator |
| magnesium stearate | 9.000 | Lubricant |
| crospovidone | 18.000 | Disintegrant |
| Tablet Film: | | |
| Hypromellose | 11.383 | Film former |
| Glyceryl tristearate | 1.138 | Plasticizer |
| Titanium dioxide (E 171) | 0.783 | Coloring agent |
| Talc | 3.131 | Coating agent |
| Ferric oxide, red (E 172) | 1.565 | Coloring agent |

The extract is mixed with 15.000 mg silica during the drying process, which yields an extract consisting of 96% by weight of the ingredients of the extract and 4% of silica. This resulting mixture is mixed with the remaining excipients of the tablet core and compressed on a suitable tablet press.

The compression forces which are needed to produce tablets of suitable breaking resistance and hence with the required breakdown times are dependent on the shapes and sizes of the punching tools used. Compression forces in the range from 2 kN to 20 kN are preferred. Higher compression forces may lead to tablets with a delayed released of active substance. Lower compression forces may produce mechanically unstable tablets. The tablet cores may have different shapes; the preferred shapes are round biplanar or biconvex and oval or oblong forms.

The coating solution is prepared by mixing the film-forming agent with the coloring materials and a plasticizer in water. Using a suitable coating pan, the film-coating solution is applied on to the tablet cores.

Preferably the tablets have an oblong shape to facilitate swallowing. In the case of a film-coated tablet containing 360 mg of extract and an extract to excipient ratio as indicated before, an oblong tablet may be about 17 mm to 18 mm long and have a width of about 8 to 9 mm. These film coated tablets of Table A are hereinbelow coded “AS 195”.

To enhance the blood circulation and/or the oxygen supply of the lower extremities, the tablet should be taken in dosages corresponding to 150 mg and 1000 mg of extract, preferably 300 mg to 800 mg, in particular, 350 mg to 750 mg daily. The total amount of extract may be divided up in 1 to 3 film coated tablets a day. The daily dose should be taken at once, preferably in the morning.

Impressive improvement of the symptoms can be expected within 6 weeks of continuous use. The optimum effect is maintained or amplified on longer use.

Methods

Participants

Male and female patients, 18 years of age or more, with proven CVII or CVI according to Widmer, with diagnosis confirmed and present for at least one year were enrolled (medically relevant concomitant diseases have to be absent). Patients who used drugs to alleviate their CVI symptoms within 4 weeks or were treated with theophyllin, diuretics, cardiac glycosides, ACE inhibitors, or calcium antagonists within 8 days prior to the first examination were not allowed to be enrolled. Compression bandages or concomitant therapy for venous problems were forbidden during the participation in the trial.

Design and Procedures

The double-blind, randomized, placebo-controlled cross-over trial was run according to the principles of the Declaration of Helsinki and the International Conference of Harmonization of Good Clinical Practice.

Each patient participated for 17 weeks in the trial: for a one-week wash-out (placebo-treated), for a 6-week treatment period (Group 1 starting with AS 195, Group 2 starting with placebo), for a 4-week wash-out (placebo-treated), and for a second 6-week treatment period (Group 1 continuing with placebo, Group 2 continuing with the AS 195 (film-coated tablets containing 360 mg dry extract of red vine leaves) or placebo tablets were taken according to the randomization schedule as single dose in the morning. Both tablets were identical with respect to size, shape, weight, inner appearance, and taste.

For laser Doppler flowmetry, the equipment was provided by LMTB, Berlin, Germany (e.g., K. Doerschel and G. Mueller, Velocity Resolved Laser Doppler Flow Measurement in Skin, Lasermedizin 1996, 12:163-171). The equipment was a computer-based mobile unit using a laser frequency of 785 nm. The laser probe was fixed 3.5 cm distal to the inner ankle of the more affected leg. After 30 minutes sitting for adaptation to room temperature, measurement started after 10 minutes standing (256 points of measurement, duration of measurement: approximately 0.4 seconds). The back-scattered light was retrieved by two diodes in the range of frequencies between 0.2 kHz to 37.2 kHz. The data were processed using a Fast Fourier Transformation (FFT). Finally, the output referred to the range of frequencies between 0.2 kHz to 10.0 kHz for vessels in the reticular venous plexus (larger mainly thermoregulative vessels, diameter more than 30 micrometer) and to the range of frequencies between 10.1 kHz to 37.2 kHz for capillaries in the subpapillary venous plexus (superficial small nutritive vessels, diameter: 6 to 30 micrometer). Transcutaneous oxygen pressure (tcPO₂) was measured using modified Clark-type polarographic electrodes containing noble metal cathodes and silver/sliver chloride anodes (TCM 3, Radiometer Copenhagen, Brønshøj, Denmark). A heating element adjacent to the anode maintained skin temperature at 43° C. At this temperature, the arterioles are maximally dilated and tcPO₂ approximates the PO₂ of arterial blood (e.g., A. Bollinger, K. Jäger, M. Jünger, and H. Seifert, The Vascular Laboratory: Advances in Non-Invasive Techniques, World J. Surg. 1988, 12:724-731).

The electrode was attached to the skin surface by an adhesive ring device which was filled with physiological saline, 3.5 cm anteriolateral from the laser Doppler probe. After 30 minutes sitting for adaptation to room temperature, measurement started after 10 minutes of standing. A measurement lasted approximately 15 minutes. The tcPO₂ values are expressed in millimeter mercury column (mmHg). Normal values available for the dorsum of the foot of patients without CVI are ranged between 40 mmHg and 80 mmHg.

Local skin temperature was measured with a thermistor fixed adjacent to the oxygen electrode in the perimalleolar region. In order to minimize effects on the skin perfusion, LDF and tcPO₂ measurements were conducted between 28° C. and 32° C. local skin temperature.

Calf and ankle circumference were measured using a measuring tape. Measurements were carried out at the lateral and medial ankle and at the middle of the calf.

Subjective symptoms of CVI (tired heavy legs, sensation of tension, tingling sensation, and pain) were measured by using a 10-cm visual analogue scale with zero as “none at all” and 10 cm as “very strong”.

Overall treatment efficacy was rated by patients and investigators on a 4-point verbal rating scale (good, satisfactory, not satisfactory, and bad) at the end of each treatment period.

Overall tolerability was rated by patients and investigators on a 4-point verbal rating scale (good, satisfactory, not satisfactory, and bad). The patients were questioned about their well-being in general terms at each visit.

Laboratory safety screens (hematology, clinical chemistry, and urinalysis) and general physical examinations were performed two times during the study. Blood pressure and heart rate while sitting were measured at each visit.

Results

Seventy-one women and men aged between 32 and 76 years with proven CVI stage I and II according to Widmer were included. The mean age (±standard deviation) was 55.2±7.7 years; 55 were women, 16 men. The phlebological status revealed moderate or severe intensity of varicosis in 47 (67.1%), pigmentation in 27 (38.6%), ankle edema in 26 (37.1%), and lower leg edema in 25 (35.7%) patients. Mild signs of atrophy were present in 13 patients (18.6%), of eczema in none (Table 1).

TABLE 1
Demographics and Baseline Characteristics of CVI

| | AS 195/Placebo | | Placebo/AS 195 | |
|---|---|---|---|---|
| | (n = 36) | | (n = 35) | |
| Continuous Variates | | | | |
| (median (range)) | | | | |
| Age [years] | 66 | (32-76) | 66 | (37-76) |
| Height [cm] | 168 | (150-186) | 165 | (150-191) |
| Weight [kg] | 76.5 | (48-97) | 73 | (55-120) |
| Body mass index [kg/m²] | 27.6 | (20.6-32.0) | 26.7 | (20.1-42.5) |
| Systolic blood pressure | 130 | (100-150) | 135 | (120-140) |
| [mmHg] | | | | |
| Diastolic blood pressure | 80 | (60-90) | 80 | (65-90) |
| [mmHg] | | | | |
| Categorical Variates (n (%)) | | | | |
| Female | 24 | (66.7) | 31 | (88.6) |
| Current smoker | 4 | (11.1) | 1 | (2.9) |
| CVI stage | | | | |
| Stage II | 26 | (72.2) | 23 | (65.7) |
| Stage II | 10 | (27.8) | 12 | (34.3) |
| Phlebological status of | | | | |
| moderate to severe intensity | | | | |
| Varicosis | 26 | (72.2) | 22 | (62.9) |
| Pigmentation | 11 | (30.6) | 17 | (48.6) |
| Atrophy | 0 | (0.0) | 0 | (0.0) |
| Eczema | 0 | (0.0) | 0 | (0.0) |
| Ankle edema | 13 | (36.1) | 14 | (40.0) |
| Lower leg edema | 12 | (33.3) | 14 | (40.0) |

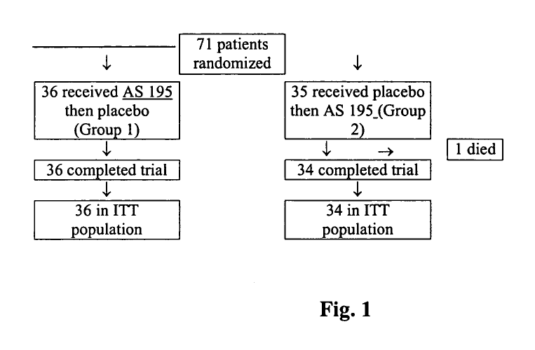

One 76 year old man died from a heart attack during a tennis match (while on placebo). This patient was excluded from the intention-to-treat analyses. Protocol violations did not occur in the remaining patients. Therefore, all 70 patients remained in the intention to treat analyses (FIG. 1). Patient characteristics were homogenously distributed across the two treatment sequences (Group 1, Group 2), except for the sex ratio (12 men in Group 1, 4 men in Group 2) (Table 1). Baseline values for the laser Doppler parameters, transcutaneous oximetry, ankle and calf circumferences, and subjective symptoms were comparable for Group 1 and Group 2 (Table 2). Compliance was approximately 100% in both treatment sequences.

TABLE 2
Mean (±SD) of Baseline Characteristics of Each Treatment Period

| | Period 1 | | Period 2 | |
|---|---|---|---|---|
| | AS 195 | Placebo | AS 195 | Placebo |
| | (n = 36) | (n = 34) | (n = 34) | (n = 36) |
| Laser Doppler Flowmetry | | | | |
| [AU] | | | | |
| 10-37 kHz | 303.5 (135.2) | 333.5 (153.0) | 275.4 (126.4) | 293.3 (119.9) |
| <10 kHz | 352.7 (87.7) | 370.8 (120.0) | 174.7 (77.0) | 189.4 (67.6) |
| Transcutaneous Oximetry | 32.1 (7.0) | 32.3 (6.4) | 30.1 (6.2) | 30.8 (6.4) |
| [mmHg] | | | | |
| Circumference [cm] | | | | |
| Ankle | 20.3 (2.2) | 20.4 (2.4) | 20.2 (2.6) | 20.3 (2.2) |
| Calf | 34.7 (3.1) | 34.2 (3.0) | 34.0 (3.1) | 34.6 (3.2) |
| Subjective symptoms [cm] | | | | |
| Tired/heavy legs | 4.3 (2.8) | 3.7 (2.9) | 4.6 (2.9) | 5.2 (2.6) |
| Pain in legs | 4.0 (3.2) | 3.2 (3.1) | 4.5 (2.7) | 4.9 (3.1) |
| Sensation of tension | 4.5 (2.9) | 4.1 (2.8) | 4.5 (2.6) | 5.1 (2.5) |
| Tingling sensation | 3.3 (3.1) | 2.7 (2.9) | 3.7 (2.6) | 4.2 (2.8) |

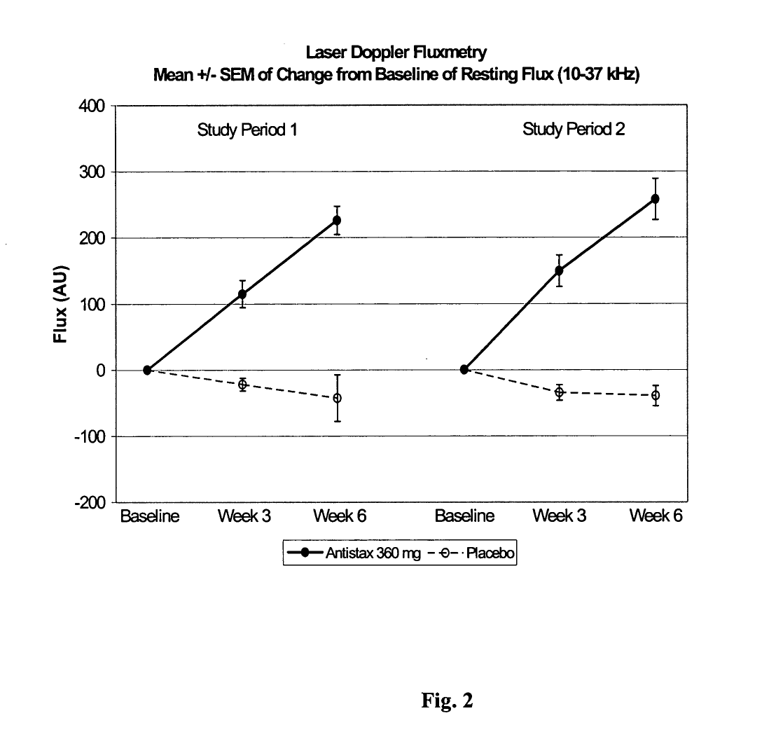

Laser Doppler flow measurements in the frequency range of 10-37 kHz were elected for the primary endpoint. These frequencies are considered to be determined by the number of erythrocytes and their movements (flow velocity) in the capillaries of the superficial layer of the skin of the leg. After 6 weeks, the laser Doppler frequencies (10-37 kHz) increased in the AS 195 group (+241.8±18.7 AU) but decreased in the placebo group (−41.0±18.7 AU, p<0.0001) (Table 3). This effect was present as early as 3 weeks after start of treatment (p<0.0001) (Table 4, FIG. 2).

TABLE 3
Mean (±SEM) of Change from Baseline Adjusted for Period Effects,
95% Confidence Interval for Treatment Contrasts and p Value after 3
Weeks Treatment with 360 mg AS 195 or Placebo

| | |
|---|---|
| Treatment | Treatment contrast |

| | AS 195 | Placebo | Difference | interval | |
|---|---|---|---|---|---|
| | (n = 70) | (n = 70) | (n = 70) | (n = 70) | p value |
| Week 3 | | | | | |
| Laser Doppler | | | | | |
| Flowmetry [AU] | | | | | |
| 10-37 kHz | 132.2 (11.9) | −28.2 (11.9) | 160.5 | 127.0 to 194.0 | <0.0001 |
| <10 kHz | −3.7 (9.2) | −99.9 (9.2) | 96.2 | 70.2 to 122.2 | <0.0001 |
| Transcutaneous | 0.62 (0.97) | −3.84 (0.97) | 4.46 | 1.72 to 7.20 | 0.0018 |
| Oximetry [mmHg] | | | | | |
| Circumference | | | | | |
| [cm] | | | | | |
| Ankle | −0.19 (0.09) | 0.21 (0.09) | −0.40 | −0.65 to −0.15 | 0.0025 |
| Calf | −0.24 (0.04) | 0.04 (0.04) | −0.28 | −0.40 to −0.17 | <0.0001 |
| Subjective | | | | | |
| symptoms | | | | | |
| [cm] | | | | | |
| Tired/heavy legs | −0.94 (0.25) | 0.21 (0.25) | −0.73 | −1.42 to −0.04 | 0.0396 |
| Pain in legs | −1.17 (0.23) | −0.24 (0.23) | −0.94 | −1.59 to −0.28 | 0.0061 |
| Sensation of | −1.00 (0.24) | −0.52 (0.24) | −0.49 | −1.17 to 0.19 | 0.1588 |
| tension | | | | | |
| Tingling | −0.99 (0.26) | −0.20 (0.26) | −0.79 | −1.52 to −0.06 | 0.0335 |
| sensation | | | | | |

TABLE 4
Mean (±SEM) of Change from Baseline Adjusted for Period Effects,
95% Confidence Interval for Treatment Contrasts and p Value after 6 Weeks
Treatment with 360 mg AS 195 or Placebo

| | | | Treatment contrast | | |
|---|---|---|---|---|---|
| | Treatment | | | Confidence | |
| | AS 195 | Placebo | Difference | interval | |
| | (n = 70) | (n = 70) | (n = 70) | (n = 70) | p value |
| Week 6 | | | | | |
| Laser Doppler | | | | | |
| Flowmetry [AU] | | | | | |
| 10-37 kHz | 241.8 (18.7) | −41.0 (18.7) | 282.8 | 229.9 to 335.7 | <0.0001 |
| (primary | | | | | |
| endpoint) | | | | | |
| <10 kHz | 57.0 (12.4) | −107.7 (12.4) | 164.7 | 129.7 to 199.7 | <0.0001 |
| Transcutaneous | 1.35 (0.97) | −7.27 (0.97) | 8.63 | 5.88 to 11.38 | <0.0001 |
| Oximetry [mmHg] | | | | | |
| Circumference | | | | | |
| [cm] | | | | | |
| Ankle | −0.39 (0.09) | 0.29 (0.09) | −0.68 | −0.94 to −0.43 | <0.0001 |
| Calf | −0.54 (0.05) | 0.14 (0.05) | −0.68 | −0.83 to −0.53 | <0.0001 |
| Subjective | | | | | |
| symptoms | | | | | |
| [cm] | | | | | |
| Tired/heavy legs | −0.78 (0.33) | −0.94 (0.33) | 0.16 | −0.76 to 1.09 | 0.7285 |
| Pain in legs | −0.76 (0.35) | −0.86 (0.35) | 0.10 | −0.88 to 1.09 | 0.8323 |
| Sensation of | −0.96 (0.35) | −1.40 (0.35) | 0.44 | −0.46 to 1.44 | 0.3819 |
| tension | | | | | |
| Tingling sensation | −0.55 (0.30) | −0.66 (0.30) | 0.11 | −0.75 to 0.96 | 0.8044 |

Laser Doppler flow measurements in the frequency range below 10 kHz are considered to be determined by the number of erythrocytes and their movements (flow velocity) in the capillaries in the deeper mainly thermoregulative layer of the skin of the leg. After 6 weeks the laser Doppler frequencies below 10 kHz) increased in the AS 195 group (+57.0±12.4 AU) and decreased in the placebo group (−107.7±12.4 AU, p<0.0001) (Table 3). This effect seems to depend on the climatic condition during the treatment period. During the study period of moderate temperatures (April/May), the laser Doppler measurements (<10 kHz) remained unchanged in the AS 195 treatment group after an initial drop, whereas the measurements in the placebo group decreased (p<0.0001). During the study period of higher temperatures (July/August), the laser Doppler measurements (<10 kHz) increased in the AS 195 treatment group and remained constant in the placebo group. (p<0.0001).

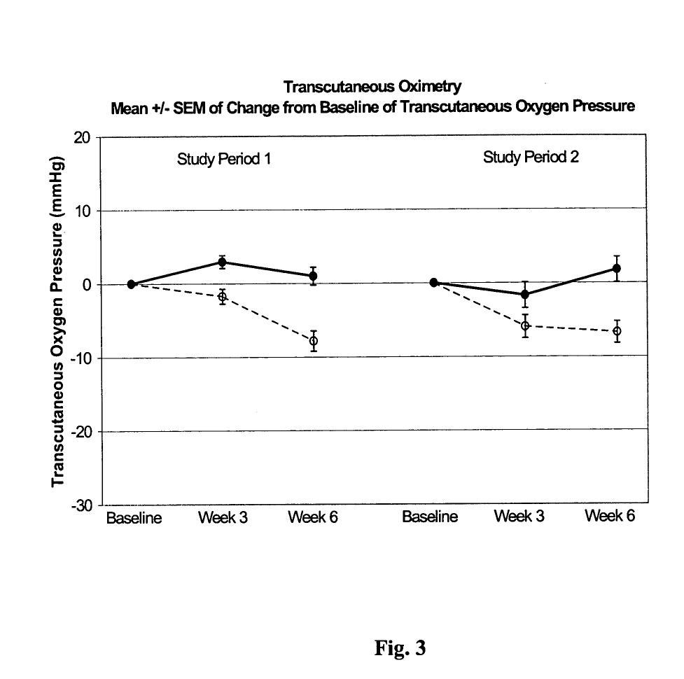

The transcutaneous oxygen pressure increased in the AS 195 group (+1.35±0.97 mmHg) but decreased in the placebo group (−7.27±0.97 mmHg, p<0.0001). This observation was consistent in both treatment periods and would therefore be in line with the laser Doppler flow in the nutritive superficial layer of the skin (i.e., 10-37 kHz) (Table 3,4, FIG. 3).

The statistically significant and clinically relevant reduction of ankle (after 3 weeks: AS 195: −0.19±0.09 cm, placebo +0.21±0.09 cm, p=0.0025) and calf circumferences (after 3 weeks: AS 195: −0.24±0.04 cm, placebo +0.04±0.04 cm, p<0.0001) indicate an onset of action as early as 3 weeks after start of treatment (Table 3). This effect becomes more pronounced after 6 weeks (AS 195 ankle: −0.39±0.09 cm, calf: −0.54±0.05; placebo ankle: +0.29±0.09 cm, calf: +0.14±0.05 cm, p<0.000 1) (Table 4)

There was no relevant change of the intensity of the subjective symptoms related to CVI after 6 weeks of treatment. This result is in line with those of a previous study where subjective symptoms measured on a visual analogue scale were reduced only after longer treatment periods (12 weeks).

Adverse events occurred rarely in this study. Thirteen of 71 patients experienced at least one adverse event, 12 of them experienced the onset of action while on placebo treatment, one while on AS 195 (bronchitis, moderate intensity, considered not drug related by the investigator). The patient who died from cardiac arrest had been treated with placebo (never received AS 195 in this trial). All patients assessed the overall tolerability as good or satisfactory. The laboratory parameters did not change during the study.

Discussion

It has been shown in a previous study (WO 01/28363) that red vine leaves extract AS 195 reduces lower leg edema, calf circumference, and ankle circumference in addition to improving subjective symptoms related to chronic venous insufficiency in patients treated once daily for 12 weeks. The present study was designed to provide additional information on the underlying mechanism of action by investigating microcirculation as a clinically relevant surrogate parameter for CVI related leg problems. This study is the first one in CVI patients aimed to investigate, in addition to leg edema reduction, further clinical relevant effects related to the therapy with red vine leaves extract. The reduced venous drainage results in impaired cutaneous microcirculation with trophical disturbances of the skin. If CVI remains untreated this condition may even result venous leg ulcers. Laser Doppler flowmetry, as used in the present study, is a valid and sensitive method to measure objective treatment effects which may be related to the subjectively experienced volume reduction after 3 months of treatment.

The study results fit into the clinical data available for AS 195 and add information on the onset of action. The leg volume as an objective parameter will be reduced in a clinically relevant and statistically significant degree after 6 weeks of treatment. This objective effect has also been reported recently with horse chestnut seed extract (e.g., C. Diehm, H. J. Trampisch, S. Lange, and C. Schmidt, Comparison of Leg Compression Stocking and Oral Horse-Chestnut Seed Extract Therapy in Patients with Chronic Venous Insufficiency, Lancet 1996, 347:292-294) and Butcher's Broom (e.g., W. Vanscheidt, V. Jost, P. Wolna, et al., Efficacy and Safety of a Butcher's Broom Preparation (Ruscus aculeatus L. extract) Compared to Placebo in Patients Suffering from Chronic Venous Insufficiency, Drug Res. 2002, 52(4):243-250).

In the present study it was shown that the laser Doppler flowmetry parameters, the ankle and calf circumferences and the transcutaneous oxygen pressure were affected as early as after 3 weeks of treatment. In contrast, the subjective symptoms of CVI rated on a visual analogue scale were not significantly different from placebo after 6 weeks of treatment as they were in the previous study. A treatment duration of 12 weeks is mandatory for a relevant reduction of subjective CVI symptoms.

The present results suggest a major role of red vine leaves extract in prevention of CVI progression and the occurrence of trophical skin lesions and may even prevent or delay the transition from clinically not relevant early stages of CVI to CVI Stage I.

We claim:

1. A film coated tablet comprising:
(a) at least 50% by weight of a dried aqueous extract of red vine leaves;
(b) up to 50% by weight of an excipient consisting essentially of: at least one binder, at least one disintegrant, at least one filler, and a lubricant; and
(c) a tablet film consisting essentially of: a film former, a plasticizer, a coating agent, and optionally a coloring agent.

2. The film coated tablet of claim 1, wherein the dried aqueous extract of vine leaves further comprises an addition of up to 10% by weight of silica based on total amount of component (a).

3. The film coated tablet of claim 2, wherein the silica is colloidal, anhydrous silica.

4. The film coated tablet according to claim 1, comprising:
(a) 50% to 70% by weight of the dried aqueous extract of red vine leaves;
(b) 25% to 49% by weight of the excipient; and
(c) 1% to 5% by weight of the tablet film,
based on the total mass of the film coated tablet.

5. The film coated tablet according to claim 1, comprising:
(a) 51% to 59% by weight of the dried aqueous extract of red vine leaves;
(b) 38% to 48% by weight of the excipient; and
(c) 1 to 3% by weight of the tablet film,
based on the total mass of the film coated tablet.

6. The film coated tablet according to one of claims 1 to 5, wherein the excipient (b) consists essentially of: 70% to 85% by weight of at least one binder, 0.5% to 12.5% by weight of at least one disintegrant, 5% to 15% by weight of at least one filler, and 1% to 5% by weight of at least one lubricant, based on the total mass of the excipient (b).

7. The film coated tablet according to one of claims 1 to 5, wherein the binder is powdered cellulose, microcrystalline cellulose, starch, polyvinylpyrrolidone, copolymers of vinylpyrrolidone with other vinyl derivatives, cellulose derivatives, or a mixture thereof.

8. The film coated tablet according to claim 6, wherein the binder is powdered cellulose, microcrystalline cellulose, starch, polyvinylpyrrolidone, copolymers of vinylpyrrolidone with other vinyl derivatives, cellulose derivatives, or a mixture thereof.

9. The film coated tablet according to one of claims 1 to 5, wherein the disintegrant is colloidal silica, sodium starch glycolate, crosslinked polyvinylpyrrolidone (crospovidone), croscarmellose sodium salt (sodium salt of cellulose carboxymethyl ether, crosslinked), sodium-carboxymethylcellulose, dried maize starch, or a mixture thereof.

10. The film coated tablet according to claim 6, wherein the disintegrant is colloidal silica, sodium starch glycolate, crosslinked polyvinylpyrrolidone (crospovidone), croscarmellose sodium salt (sodium salt of cellulose carboxymethyl ether, crosslinked), sodium-carboxymethylcellulose, dried maize starch, or a mixture thereof.

11. The film coated tablet according to one of claims 1 to 5, wherein the filler is an inorganic phosphate or hydrogen phosphate.

12. The film coated tablet according to claim 6, wherein the filler is an inorganic phosphate or hydrogen phosphate.

13. The film coated tablet according to one of claims 1 to 5, wherein the filler is silicon dioxide, talc, stearic acid, sodium stearyl fumarate, magnesium stearate, or glycerol tribehenate.

14. The film coated tablet according to claim 6, wherein the filler is silicon dioxide, talc, stearic acid, sodium stearyl fumarate, magnesium stearate, or glycerol tribehenate.

15. The film coated tablet according to one of claims 1 to 5, wherein the tablet film (c) consists essentially of: 50% to 85% by weight of at least one film former, 5% to 10% by weight of at least one plasticizer, 10% to 20% by weight of at least one coating agent, and 0% to 15% by weight of at least one colorant, based on the total mass of the tablet film (c).

16. The film coated tablet according to claim 6, wherein the tablet film (c) consists essentially of: 50% to 85% by weight of at least one film former, 5% to 10% by weight of at least one plasticizer, 10% to 20% by weight of at least one coating agent, and 0% to 15% by weight of at least one colorant, based on the total mass of the tablet film (c).

17. A process for preparing a film coated tablet comprising:
(a) mixing a dried aqueous extract of red vine leaves with excipients, optionally in the presence of a volatile diluent;
(b) optionally screening the mixture obtained;
(c) compressing the mixture with a suitable tablet press; and
(d) coating the resulting tablet with a tablet film.

18. A method for making a film coated tablet containing a dried aqueous extract of red vine leaves comprising:
(a) extracting red vine leaves with water and drying the extract to obtain a dried aqueous extract of red vine leaves;
(b) combining the dried aqueous extract of red vine leaves with an excipient consisting essentially of: at least one binder, at least one disintegrant, at least one filler, and a lubricant;
(c) forming a tablet of the mixture obtained from step (b); and
(d) coating the tablet with a tablet film consisting essentially of: a film former, a plasticizer, a coating agent, and optionally a coloring agent to obtain a film coated tablet.

19. The method of claim 18, wherein the dried aqueous extract of vine leaves further comprises an addition of up to 10% by weight of silica based on total amount of the dried aqueous extract of red vine leaves.

20. The method of claim 19, wherein the silica is colloidal, anhydrous silica.

21. The method of claim 20, wherein the colloidal, anhydrous silica is added to the dried aqueous extract of vine leaves during drying or before admixing with the other constituents.

22. A method for making an aqueous extract of red vine leaves comprising:
(a) collecting red vine leaves at a point of time when the content in flavonoids has reached an optimum;
(b) drying and crushing the leaves;
(c) cutting the leaves to pieces;
(d) extracting the leaves with water at elevated temperatures for 6 to 10 hours; and
(e) concentrating and drying the aqueous extract of red vine leaves.

23. The method according to claim 22, further comprising adding during the drying process (e) up to 10% by weight of a flow regulator based on the final total amount of the aqueous extract of red vine leaves.

24. The method according to claim 23, wherein the flow regulator is silica.

25. The method according to claim 23, wherein the flow regulator is colloidal, anhydrous silica.

26. The method according to claim 25, wherein the colloidal, anhydrous silica comprises 2.5% to 7.5% by weight based on the final total amount of the aqueous extract of red vine leaves.

27. The method according to claim 26, wherein the colloidal, anhydrous silica comprises about 4% by weight based on the final total amount of the aqueous extract of red vine leaves.

28. The method according to claim 22, wherein the leaves in step (d) are extracted with water at temperatures from 60° C. to 80° C.
